(19) United States
(12) Patent Application Publication
(10) Pub. No.: US 2007/0203536 A1
(43) Pub. Date: Aug. 30, 2007

(54) Tinnitus Suppressing Cochlear Implant

(76) Inventors: Ingeborg Hochmair, Axams (AT); Martin Zimmerling, Patsch (AT)

Correspondence Address: BROMBERG & SUNSTEIN LLP, 125 SUMMER STREET, BOSTON, MA, 02110-1618, US

(21) Appl. No.: 11/671,636

(22) Filed: Feb. 6, 2007

Related U.S. Application Data
(60) Provisional application No. 60/765,775, filed on Feb. 7, 2006.

(51) Int. Cl. A61N 1/36

(52) U.S. Cl. 607/57

(57) ABSTRACT

An implantable device and corresponding method for suppression of tinnitus are described. An implantable signal processing module develops a stimulation signal for application to audio sensing tissue of the user. The signal processing module includes a tinnitus suppression mode in which the stimulation signal is unrelated to environmental sound near the user.

This application claims priority from U.S. Provisional Application 60/765,775, filed Feb. 7, 2006, the contents of which are incorporated herein by reference.

### FIELD OF THE INVENTION

The invention relates to an implantable device for persons suffering from tinnitus.

### BACKGROUND ART

A normal ear transmits sounds as shown in FIG. 1 through the outer ear 101 to the eardrum 102, which moves the bones of the middle ear 103, which in turn excites the cochlea 104. The cochlea 104 includes an upper channel known as the scala vestibuli 105 and a lower channel known as the scala tympani 106, which are connected by the cochlear duct 107. In response to received sounds transmitted by the middle ear 103, the fluid filled scala vestibuli 105 and scala tympani 106 function as a transducer to transmit waves to generate electric pulses that are transmitted to the cochlear nerve 113, and ultimately to the brain.

Some persons have partial or full loss of normal sensorineural hearing. Cochlear implant systems have been developed to overcome this by directly stimulating the user's cochlea 104. A typical system may include an external microphone that provides an audio signal input to an external signal processing stage (not shown in FIG. 1) where various signal processing schemes can be implemented. The processed signal is then converted into a digital data format, such as a sequence of data frames, for transmission into receiver 108. Besides extracting the audio information, the receiver 108 also performs additional signal processing such as error correction, pulse formation, etc., and produces a stimulation pattern (based on the extracted audio information) that is sent through connected wires 109 to an implanted electrode carrier 110. Typically, this electrode carrier 110 includes multiple electrodes on its surface that provide selective stimulation of the cochlea 104.

Existing cochlear implant systems need to deliver electrical power from outside the body through the skin to satisfy the power requirements of the implanted portion of the system. FIG. 1 shows a typical arrangement based on inductive coupling through the skin to transfer both the required electrical power and the processed audio information. As shown in FIG. 1, an external primary coil 111 (coupled to the external signal processor) is placed on the skin adjacent to a subcutaneous secondary coil 112 (connected to the receiver 108). Often, a magnet in the external coil structure interacts a corresponding magnet in the subcutaneous secondary coil structure. This arrangement inductively couples a radio frequency (rf) electrical signal to the receiver 108. The receiver 108 is able to extract from the rf signal both the audio information for the implanted portion of the system and a power component to power the implanted system.

Many profoundly deaf persons do not need or want a cochlear implant system to improve their communication skills. The implant-aided input is very different from the input via a normal ear, and therefore many profoundly deaf users do not bother to undergo any training. Therefore they do not see any advantage and soon terminate the use of their cochlear implant. In addition, cochlear implants are normally not considered for subjects suffering from unilateral hearing loss (hearing loss on one side).

Besides hearing loss, another rather depressing hearing-related affliction is tinnitus. Tinnitus is defined by the perception of a continuous ringing or beating sound without external source. This sensation can be extremely annoying and often interferes with normal daily activities including sleep.

### SUMMARY OF THE INVENTION

Embodiments of the present invention include an implantable device for suppression of tinnitus. An implantable signal processing module develops a stimulation signal for application to audio sensing tissue of the user. The signal processing module includes a tinnitus suppression mode in which the stimulation signal is unrelated to environmental sound near the user.

In further embodiments, the stimulation signal may be significantly imperceptible to the user. The device may be a cochlear implant, for example, wherein the stimulation signal is an electrical stimulation signal and may be further adapted to stimulate the scala tympani and/or scala vestibuli of the user. The device may include an implantable stimulator which may be atraumatically insertable so as to preserve residual hearing in the implanted ear. Embodiments may also include an acoustic-mechanical stimulation module for developing and acoustic-mechanical stimulation signal such that the implanted ear receives both an electrical stimulation signal and an acoustic-mechanical stimulation signal.

FIG. 2 shows an implantable system according to one specific embodiment starting from the prior art system of FIG. 1.

The electrical stimulation signal may include sequences of electrical pulses at or near a threshold level of detectability to stimulate the audio sensing tissue. The electrical pulses may have amplitudes according to a CIS-strategy threshold and may occur at rates between 10 and 10,000 pulses per second.

In other embodiments, the device may be a brainstem implant. In other embodiments, the stimulation signal may be mechanical, for example, the device may be a middle ear implant such as a floating mass transducer.

In any such embodiment, the tinnitus suppression mode may be user controllable and/or software controllable, for example, controlled by time such that the tinnitus suppression mode is time dependent. The signal processing module may further provide signal processing to provide sound localization information.

Embodiments of the present invention also include a method for tinnitus suppression. In such embodiments, a stimulation signal unrelated to environmental sound near the user is applied to audio sensing tissue of a user. The stimulation signal may not be significantly perceptible to the user.

In some embodiments, the stimulation signal is an electrical stimulation signal provided by a cochlear implant. Further, the audio sensing tissue may include the scala tympani and/or the scala vestibuli of the user. The electrical stimulation signal may be applied using an atraumatically-inserted electrode which preserves residual hearing in the implanted ear.

Embodiments may also include providing acoustic mechanical stimulation to the implanted ear, such that the implanted ear receives both an electrical stimulation signal and an acoustic-mechanical stimulation signal. Applying the electrical stimulation signal may include applying sequences of electric pulses having amplitudes according to a CIS-strategy threshold, for example, at rates between 10 and 10,000 pulses per second.

In some embodiments, the stimulating may be produced by a brainstem implant or by a middle ear implant such as a floating mass transducer.

The stimulating may be user controllable and/or software controllable, for example, to be time dependent. The stimulation signal may further provide sound localization information.

### BRIEF DESCRIPTION OF THE DRAWINGS

FIG. 1 shows the ear structure of a human ear and a typical cochlear implant system according to the prior art.

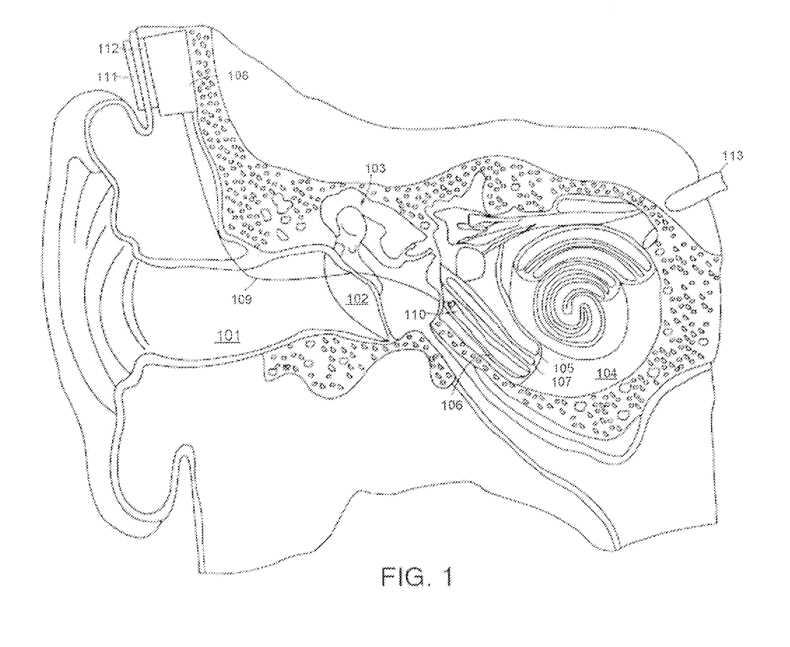

FIG. 2 shows an implantable system according to one specific embodiment of the present invention, starting from the prior art system of FIG. 1.

FIG. 3 shows a brainstem implant according to an alternative embodiment of the present invention.

### DETAILED DESCRIPTION OF SPECIFIC EMBODIMENTS

As used herein, the term “hearing implant” includes cochlear implants (CI), brainstem implants, and middle ear implants (MEI).

When stimulating, hearing implants can suppress some forms of tinnitus such as peripheral origin-tinnitus, i.e. tinnitus connected to hearing problems. Both mechanical (acoustic) and electrical stimulation have been found to provide such benefit. Specifically, unilateral tinnitus resulting from unilateral cochlear profound sensory-neural hearing loss (SNHL) can be treated with cochlear implantation, which also typically improves the overall quality of hearing in a significant way. As explained above, unilateral deafness has not previously been an indication for a cochlear implant. Thus, embodiments of the present invention include a cochlear implant that restores hearing by changing cortical activity by electrical stimulation of the auditory cortex or auditory nerve. Tinnitus suppression is realized by use of comparatively low level stimulation (“background stimulation”) at or near the hearing threshold.

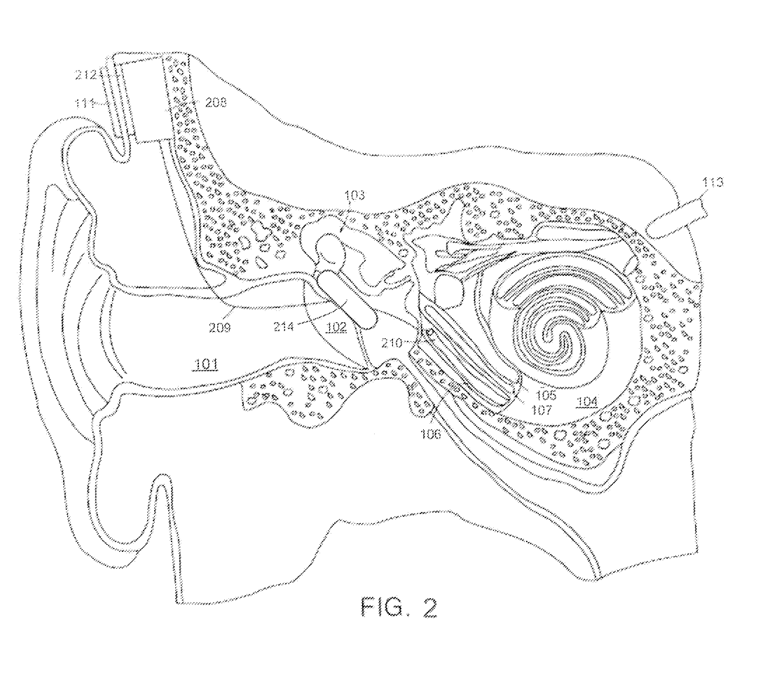

In specific embodiments, a cochlear implant may provide an electrical stimulation signal that stimulates the scala tympani and/or scala vestibuli of the user. One specific embodiment can be explained with reference to the cochlear implant system shown in FIG. 2. A stimulation signal is developed by an implantable signal processing module such as the receiver 208. An implantable electrode stimulator such as the electrode carrier 210 is coupled to the signal processing module receiver 208 for applying the stimulation signal to audio sensing tissue of a user such as the scala vestibuli 105 and scala tympani 106 of the cochlea 104. The signal processing module receiver 208 may have various operating modes which may or may not include a normal operation mode in which the stimulation signal applied to the audio sensing tissue is representative of the environmental sounds around the user.

Whether or not a normal mode is available, embodiments of the present invention include a tinnitus suppression mode in which the stimulation signal is unrelated to environmental sound near the user. In some specific embodiments, it may not be necessary to generate specific hearing sensations for tinnitus suppression, rather some low-level background stimulation appears to be sufficient. Thus, the tinnitus suppression mode in specific embodiments may be the result of switching off the microphone input, and/or providing some background stimulation at or near some threshold detection level of stimulation, such as provided by a CIS-type strategy as implemented, for example, in the Pulsar and Sonata implants by MED-EL of Innsbruck, Austria. The electrical stimulation signal may be in the form of CIS-type pulses, for example, at a rate of 10 to 10,000 pulses per second, at a pulse amplitude near the CIS threshold.

The electrode carrier 210 may be inserted relatively deeply in order to reach the low frequency response region of the cochlea 104 and thereby cover the entire frequency range. This may be especially important if the tinnitus effects occur in the low frequencies. In addition, use of such a deep insertion electrode carrier 210 in a cochlear implant for a unilaterally deaf person may also provide sound localization information so to restore or partially restore directional hearing for those persons in which the working ear is confined to low frequencies. In addition or alternatively, the electrode carrier 210 may also be suitable for atraumatic insertion so as to preserve some or all of the residual hearing in the implanted ear, which is often not a consideration in a cochlear implant for a totally deaf patient.

Some embodiments not only provide tinnitus suppression, but may also fully or partially restore bilateral hearing. This advantage is related to the use of an electrode carrier 210 adapted for deep insertion, in order to efficiently exploit the inputs from the normal ear and from the implanted signal processing module receiver 208. The improvement in bilateral hearing may in turn improve speech understanding in noisy environments. In addition, directional hearing may be fully or partially restored. These effects generally may improve over time as the user accumulates experience with the device, especially if hearing on the non-implanted ear is also compromised.

Since tinnitus can be especially annoying during sleep time, a totally implantable cochlear implant can be of great advantage (albeit not absolutely necessary) to stimulate without having to wear an external part, which would have to be securely fastened and which might be uncomfortable. The tinnitus suppression mode may be user controllable and/or software controllable. For example, an embodiment could include a clock function to switch off sound input for a selected time (while retaining background stimulation for tinnitus suppression) such as at night for sleeping, or to optionally switch off stimulation after some time, and/or to set an alarm.

If the subject retains some residual hearing in the implanted ear, the device also can be used together with an acoustic-mechanical stimulation module 214 to result in improved hearing quality and improved sound localization capability based on the application of both an electrical stimulation signal from the electrode carrier 210 and an acoustic mechanical stimulation signal from the acoustic mechanical stimulation module 214. The acoustic mechanical stimulation module 214 mechanically drives the ossicular chain, which in turn stimulates the cochlea 104. An acoustic mechanical stimulation module 214 in the specific form of a middle ear implant based on a floating mass transducer is further described, for example, in U.S. Pat. Nos. 5,913,815; 5,897,486; 5,624,376; 5,554,096; 5,456,654; 5,800,336; 5,857,958; and 6,475,134, each of which is incorporated herein by reference.

An alternative embodiment may have an acoustic mechanical stimulation module 214 with a tinnitus suppression mode, without any implanted electrode stimulation system so that only acoustic-mechanical stimulation is provided. Thus, specific embodiments may be in the form of a Middle Ear Implant (MEI) such as a “Soundbridge” (and its derivations) in which the stimulation is acoustic-mechanical via a “floating mass transducer.” The advantages gained by the patient are similar to a cochlear implant embodiment, and built-in features could be very similar as well. An MEI is often designed for moderately hearing impaired patients, who usually try to use conventional hearing aids thus avoiding surgery necessary for the MEI. However, because of the improved sound quality and the ability to suppress tinnitus during sleep, such a device may be more readily accepted.

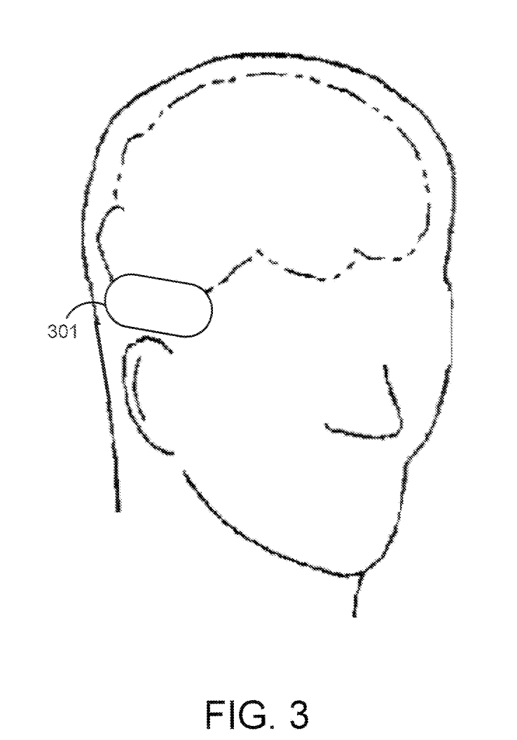

FIG. 3 shows another embodiment for tinnitus suppression in the specific form of a brainstem implant 301. In other embodiments, instead of a deep insertion scala tympani electrode, other stimulating means may be used, such as split electrodes (to stimulate the scala vestibuli), brainstem electrodes, floating mass transducer (at the ossicles or at the round window), and/or a bone bridge.

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable device for suppression of tinnitus, the device comprising:
an implantable signal processing module for developing a stimulation signal for application to audio sensing tissue of the user;
wherein the signal processing module includes a tinnitus suppression mode in which the stimulation signal is unrelated to environmental sound near the user.

2. A device according to claim 1, wherein the stimulation signal is not significantly perceptible to the user.

3. A device according to claim 1, wherein the device is a cochlear implant and the stimulation signal is an electrical stimulation signal.

4. A device according to claim 3, wherein the audio sensing tissue includes the scala tympani of the user.

5. A device according to claim 3, wherein the audio sensing tissue includes the scala vestibuli of the user.

6. A device according to claim 3, further comprising:
an implantable stimulator for applying the stimulation signal to the audio sensing tissue, the stimulator being atraumatically insertable with respect to the audio sensing tissue so as to preserve residual hearing in the implanted ear.

7. A device according to claim 6, further comprising:
an acoustic-mechanical stimulation module for developing an acoustic-mechanical stimulation signal such that the implanted ear receives both an electrical stimulation signal and an acoustic-mechanical stimulation signal.

8. A device according to claim 3, wherein the electrical stimulation signal includes sequences of electric pulses having amplitudes according to a CIS-strategy threshold.

9. A device according to claim 8, wherein the electrical pulses occur at rates between 10 and 10,000 pulses per second.

10. A device according to claim 1, wherein the device is a brainstem implant.

11. A device according to claim 1, wherein the device is a middle ear implant.

12. A device according to claim 11, wherein the middle ear implant is a floating mass transducer.

13. A device according to claim 1, wherein the tinnitus suppression mode is user controllable.

14. A device according to claim 1, wherein the tinnitus suppression mode is software controllable.

15. A device according to claim 1, wherein the tinnitus suppression mode is time dependent.

16. A device according to claim 1, wherein the signal processing module further provides signal processing to provide sound localization information.

17. A method of tinnitus suppression comprising:
applying to audio sensing tissue of a user a stimulation signal unrelated to environmental sound near the user.

18. A method according to claim 17, wherein the stimulation signal is not significantly perceptible to the user.

19. A method according to claim 17, wherein the stimulation signal is an electrical stimulation signal applied by a cochlear prosthesis.

20. A method according to claim 19, wherein the audio sensing tissue includes the scala tympani of the user.

21. A method according to claim 19, wherein the audio sensing tissue includes the scala vestibuli of the user.

22. A method according to claim 19, wherein the electrical stimulation signal is applied by an atraumatically-inserted stimulator which preserves residual hearing in the implanted ear.

23. A method according to claim 22, further comprising:
providing acoustic mechanical stimulation to the implanted ear, such that the implanted ear receives both an electrical stimulation signal and an acoustic-mechanical stimulation signal.

24. A method according to claim 19, wherein providing an electrical stimulation signal to the audio sensing tissue includes providing sequences of electric pulses having amplitudes according to a CIS-strategy threshold.

25. A method according to claim 24, wherein the electrical pulses occur at rates between 10 and 10,000 pulses per second.

26. A method according to claim 17, wherein the stimulation signal is applied by a brainstem implant.

27. A method according to claim 17, wherein the stimulation signal is applied by a middle ear implant.

28. A method according to claim 17, wherein the middle ear implant is a floating mass transducer.

29. A method according to claim 17, wherein the stimulation signal is user controllable.

30. A method according to claim 17, wherein the stimulation signal is software controllable.

31. A method according to claim 17, wherein applying the stimulation signal is time dependent.

32. A method according to claim 17, wherein applying the stimulation signal further provides sound localization information.
